(19) United States
(12) Patent Application Publication
(10) Pub. No.: US 2021/0128324 A1
(43) Pub. Date: May 6, 2021

(54) MIDSTANCE PROSTHETIC FOOT

(71) Applicant: HARIPRIYA SUMANA GOSAKAN, Bangalore (IN)

(72) Inventor: HARIPRIYA SUMANA GOSAKAN, BANGALORE (IN)

(21) Appl. No.: 17/259,542

(22) PCT Filed: Jul. 11, 2019
(86) PCT No.: PCT/IN2019/050513

§ 371 Date: Jan. 11, 2021

(30) Foreign Application Priority Data
Jul. 13, 2018 (IN) 201841026163

(51) Int. Cl. A61F 2/66

(52) U.S. Cl. CPC A61F 2/66; A61F 2002/6664

(57) ABSTRACT

The prosthetic foot (101) includes a first heel (102) and a second heel (103). The second heel (103) provides midstance stability during the mid-stage of foot motion, that is, in the stage between the heel strike and the toe-off. The prosthetic foot (101) is not limited to only two heels, but may be extended to include a multiplicity of heels.

### CROSS-REFERENCE TO RELATED APPLICATIONS

This PCT application claims priority to and the benefit of provisional patent application titled “Midstance Prosthetic Foot”, application number 201841026163, filed in the Indian Patent Office on 13 Jul. 2018. The specification of the above referenced patent application is incorporated herein by reference in its entirety.

### BACKGROUND

The apparatus disclosed herein, in general, relates to a medical device, and in particular relates to a prosthetic foot.

Amputees with a loss of a limb experience discomfort and imbalance during the midstance phase while using a prosthetic foot. Hence, there is an unmet need for providing a smooth rollover or transition during the mid-stage of foot motion, that is, in the stage between the heel strike and the toe-off. The sudden and “flat footed” sensation as a result of the sudden transition between heel strike and toe-off needs to be eliminated.

### SUMMARY OF THE INVENTION

The apparatus disclosed herein addresses the above recited unmet need for midstance stability for a prosthetic foot by providing a secondary or a multiplicity of heels. The secondary or multiplicity of heels provide a cushioning effect, in single or in multiple stages. The single or multiple heel system smoothens the transition between heel strike and toe-off, thereby providing a more comfortable experience to the amputee.

### BRIEF DESCRIPTION OF THE DRAWINGS

FIG. 1 illustrates a prosthetic foot that provides midstance stability.

### DETAILED DESCRIPTION OF THE INVENTION

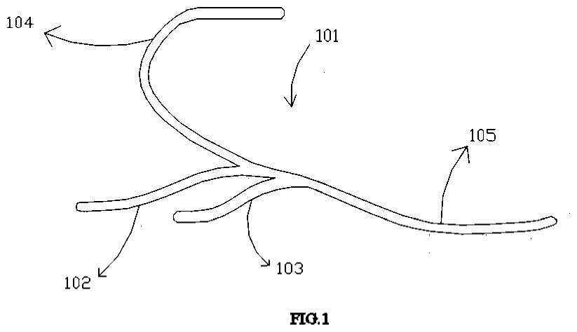

FIG. 1 illustrates a prosthetic foot 101 that provides midstance stability. The prosthetic foot 101 comprises a first heel 102 and a second heel 103. The second heel 103 provides midstance stability during the mid-stage of foot motion, that is, in the stage between the heel strike and the toe-off. The prosthetic foot 101 disclosed herein is not limited to only two heels, but may be extended to include a multiplicity of heels. The prosthetic foot 101 is manufactured using one or a combination of the following: carbon fiber composites, glass fiber composites, plastics, etc. For example, the prosthetic foot 101 comprises carbon fiber in a thermosetting or thermoplastic matrix.

The prosthetic foot comprises a top composite leaf spring 104, a first heel 102, a second heel 103. The second heel 103 and the first heel 102 are integral continual parts of lower composite leaf spring 105.

The prosthetic foot 101 is manufactured using one or a combination of the following materials: carbon fiber composites, glass fiber composites, plastics, metal, etc. For example, the prosthetic foot 101 comprises carbon fiber in a thermosetting or thermoplastic matrix.

For example, unidirectional carbon fiber prepegs may be laid in a multiaxial layup sequence, and high temperature cured in a pressurized vacuum bagged environment in a metallic mold at a high temperature of 150 Celcius.

For example, the second heel 103 may be of thickness in the range 2 mm to 5 mm, and preferably of 3 mm to 4 mm. The fiber layup sequence may comprise the following: Top woven 0/90, +45, −45, 0, 0, 0, 0, 0, 0, 0, 0−45, +45, Lower woven 0/90. The thickness of each prepeg layer may be in the 0.1 mm to 0.25 mm. To increase thickness, the number of prepeg layers may be increased.

Optionally a polyurethane insert may be introduced in the area between the carbon fiber leafsprings for an additional cushioning effect in the transition phase.

The foregoing examples have been provided merely for explanation and are in no way to be construed as limiting of the prosthetic foot 101 disclosed herein. While the prosthetic foot 101 has been described with reference to various embodiments, it is understood that the words, which have been used herein, are words of description and illustration, rather than words of limitation. Furthermore, although the prosthetic foot 101 has been described herein with reference to particular means, materials, and embodiments, the prosthetic foot 101 is not intended to be limited to the particulars disclosed herein; rather, the prosthetic foot 101 extends to all functionally equivalent structures, methods and uses, such as are within the scope of the appended claims. While multiple embodiments are disclosed, it will be understood by those skilled in the art, having the benefit of the teachings of this specification, that the prosthetic foot 101 disclosed herein is capable of modifications and other embodiments may be effected and changes may be made thereto, without departing from the scope and spirit of the prosthetic foot 101 disclosed herein.

I claim:

1. A prosthetic foot comprising:
a top composite leaf spring;
a first heel;
a second heel, wherein said second heel and said first heel are integral continual parts of a lower composite leaf spring.

2. The prosthetic foot of claim 1, wherein said first and second composite leafspring further comprise a composite fiber in a resin matrix.

3. The prosthetic foot of claim 2, wherein said composite fiber is one of or a combination of carbon fiber, aramid fiber and glass fiber.

4. The prosthetic foot of claim 2, wherein said resin matrix is one of epoxy, vinyl ester, polyester or polyurethane.
